(19) United States
(12) Patent Application Publication
(10) Pub. No.: US 2014/0217306 A1
(43) Pub. Date: Aug. 7, 2014

(54) SPLIT DRIVE ASSEMBLY AND GUIDE SLEEVES FOR UV DISINFECTION MODULES

(71) Applicants: BRUNO FERRAN, BLOOMFIELD, NJ (US); ROBERT MOLTER, FLANDERS, NJ (US); MANILEO LAPUGOT, BLOOMFIELD, NJ (US)

(72) Inventors: BRUNO FERRAN, BLOOMFIELD, NJ (US); ROBERT MOLTER, FLANDERS, NJ (US); MANILEO LAPUGOT, BLOOMFIELD, NJ (US)

(21) Appl. No.: 13/756,948

(22) Filed: Feb. 1, 2013

(51) Int. Cl. C02F 1/32; A61L 2/10

(52) U.S. Cl. CPC C02F 1/32; A61L 2/10
USPC 250/436

(57) ABSTRACT

The present invention is generally directed to a split drive assembly for an ultraviolet (UV) disinfection module comprising one or more UV lamps extending between one or more headers and a cleaning plate having openings therein arranged to substantially coincide with the UV lamps to permit movement of the cleaning plate along the UV lamps, the split drive assembly operatively connected to a moving device, the split drive assembly including a split drive nut having an internal bore for attachment to the moving device, the drive nut comprising at least two portions attached together around the moving device. Other aspects of the invention may include a sleeve guide including an attachment plate to attach the sleeve guide to a surface of the cleaning plate; a sleeve extended from the attachment plate; and an alignment device in contact with a UV lamp, the alignment device attached to the sleeve.

### BACKGROUND OF THE INVENTION

The present invention is generally directed to a cleaning system for ultraviolet (UV) disinfection modules. More specifically, the present invention is directed to a split wiper or brush, with associated housing, that permits cleaning systems to be quickly and efficiently replaced or adapted to specific treatment conditions.

Utilization of UV for disinfection of potable and wastewater increases on an ongoing basis. A number of UV disinfection systems are commercially available and utilized for a wide variety of wastewater and potable disinfection process.

In general, UV light is a portion of the electromagnetic spectrum that has many uses. For example, UV light can be used in a purification or disinfection system to kill bacteria and break down chemicals in a fluid, such as water or air. UV light can also be used to cause chemical reactions in order to break down certain chemicals and make certain chemical compounds.

In order to harness this ability of UV light, a UV reactor may be utilized. In general, a UV reactor may comprise one or more UV lights, often made from a straight hollow tube of UV transparent material, such as quartz. This tube is filled with a gas such that when an electric current passes through the gas, ultraviolet light is produced. Such UV lamps are often placed in a secondary jacket of UV transparent material, again, such as quartz. The jacket may keep water or wastewater away from the lamp. The lamp and jacket may be referred to as a reactor tube.

One or more reactor tubes may be placed in a disinfection module so that the water or wastewater flows through and/or around the reactor tubes. However, it has been an ongoing problem in the field of UV disinfection that, over the course of time, the quartz jackets surrounding the individual UV lamps tend to foul due to the slow build-up or accumulation of deposited material on the quartz jackets. Such materials may include inorganic scaling from the precipitation of metal salts, particulates, fats, oils, greases and the like that are typical of foreign matter contained with the water being disinfected. A number of systems and processes have been developed to remove such accumulations and/or deposits. Such systems include various reciprocating wiper systems which tend to have one problem or another in effectively and economically achieving the task of cleaning quartz jackets for an extended period of time.

As fouling and scale accumulate on the outer jackets, it increasingly blocks the UV light, thereby reducing the effectiveness of the disinfection module. However, oftentimes, due to the arrangement of multiple reactor tubes, the task of cleaning the jackets is difficult without partially dismantling the disinfection module. This issue has been addressed in the art in various ways, for example by U.S. Pat. Nos. 6,649,917 and 5,952,663, each owned by Infilco Degremont, Inc., and incorporated herein by reference in their entirety.

Broadly speaking, U.S. Pat. No. 6,649,917 teaches utilizing a cleaning plate with a multiplicity of holes, and associated wipers. The cleaning plate traverses between two headers along the path of the reactor tubes, and with the reactor tubes traveling through the holes and associated wipers on the cleaning plate. However, issues with the gradual decreasing effectiveness of the wipers or brushes that encircle the reactor tubes may be present. In addition, depending on specific water and/or wastewater conditions, the type of cleaning implement (e.g., a solid wiper or a brush) may vary.

Changing the wipers or brushes may be a time and manpower intensive task, as a header may need to be removed to slide the cleaning plate and wipers off of the multiplicity of lamps, replace the wipers, and slide the entire unit back on. Such time and manpower requirements reduce the ability to quickly and efficiently change the cleaning implement if the water and/or wastewater conditions change.

In addition, some prior art systems utilize a cleaning system wherein the wipers are attached to a plate, and the plate is driven substantially parallel with the lamps, for example, through the use of a hydraulic piston or threaded rod or rotatable screw. However, over time the interface between the threaded rod or rotatable screw and the cleaning plate may erode, causing the cleaning plate to either fail to move, or to be move in an uncontrolled manner, perhaps causing the cleaning plate to become wedged amongst the UV lamps.

As UV disinfection systems strive to maintain greater and greater degrees of efficiency, the cleaning plates may be utilized more often than initially designs took into account. Therefore, the lifespan of such drive nuts may be less than anticipated. Changing a drive nut attached to a cleaning plate may be a relatively time-consuming process, since at least one end of the treaded rod must be free to allow the drive nut to rotate off of the threaded rod or rotatable screw and a replacement drive nut rotated on.

Moreover, cleaning plates are often moved by a single device (such as, for example, a threaded rod or rotatable screw as discussed above). In order to maintain the cleaning plate substantially perpendicular to the UV lamps, and thereby maintain a constant cleaning pressure on the lamps without causing the cleaning plate to go askew and become jammed amongst the UV lamps, proper alignment of the cleaning plate is essential.

Accordingly, a cleaning plate system with a drive nut that can be quickly and efficiently changed, as well as a cleaning plate system that maintains proper alignment and guidance of the cleaning plate during its travels, is desired.

### SUMMARY OF THE INVENTION

In accordance with some embodiments of the present invention, aspects of the invention may comprise a split drive assembly for an ultraviolet (UV) disinfection module the UV disinfection module comprising one or more UV lamps extending between one or more headers and a cleaning plate having one or more openings therein arranged to substantially coincide with the one or more UV lamps to permit movement of the cleaning plate along the UV lamps, the split drive assembly operatively connected to a moving device, comprising: a split drive nut having an internal bore for attachment to the moving device, the drive nut comprising at least two portions attached together around the moving device.

In accordance with some embodiments of the present invention, other aspects of the present invention may comprise a split drive assembly for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the split drive assembly operatively connected to a rotatable screw, comprising: a split drive nut comprising at least two portions attached together, the split drive nut comprising: a threaded internal bore sized to engage with the rotatable screw; and an external threaded portion; an attachment plate, used to attach the split drive assembly to the cleaning plate; and a sleeve, comprising internal threads sized to engage with the external threaded portion of the split drive nut; and a guide, disposed in the sleeve and comprising a bore sized substantially similar to a diameter of the rotatable screw; wherein the attachment plate is sandwiched between the split drive nut and the sleeve when the split drive nut is inserted into and attached to the sleeve.

In accordance with some embodiments of the present invention, aspects may include a sleeve guide for a cleaning system for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the sleeve guide comprising: an attachment plate to attach the sleeve guide to a surface of the cleaning plate; a sleeve extended from the attachment plate; and an alignment device in contact with a UV lamp, the alignment device attached to the sleeve.

In accordance with some embodiments of the present invention, aspects may include a sleeve guide for a cleaning system for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the sleeve guide comprising: an attachment plate to attach the sleeve guide to a surface of the cleaning plate; a flange; a sleeve extended between the attachment plate and the flange; a top plate; and an alignment device in contact with a UV lamp, the alignment device sandwiched between the flange and the top plate.

Note that other aspects will become apparent from the following description of the invention taken in conjunction with the following drawings, although variations and modifications may be effected without departing from the spirit and scope of the novel concepts of the invention.

### BRIEF DESCRIPTION OF THE DRAWING

The present invention can be more fully understood by reading the following detailed description together with the accompanying drawings, in which like reference indicators are used to designate like elements. The accompanying figures depict certain illustrative embodiments and may aid in understanding the following detailed description. Before any embodiment of the invention is explained in detail, it is to be understood that the invention is not limited in its application to the details of construction and the arrangements of components set forth in the following description or illustrated in the drawings. The embodiments depicted are to be understood as exemplary and in no way limiting of the overall scope of the invention. Also, it is to be understood that the phraseology and terminology used herein is for the purpose of description and should not be regarded as limiting. The detailed description will make reference to the following figures, in which:

FIG. 1 illustrates a top view of a UV disinfection module, as known in the prior art.

FIG. 2 depicts an exemplary cleaning plate, used to clean the reactor tubes in a UV disinfection module, as known in the prior art.

FIG. 3 illustrates a UV disinfection module with a split drive nut, in accordance with some embodiments of the present invention.

FIG. 4 illustrates a split drive nut in accordance with some embodiments of the present invention.

FIG. 5 illustrates a split drive assembly in accordance with some embodiments of the present invention.

FIG. 6 illustrates a UV disinfection module with a split drive nut and sleeve guides, in accordance with some embodiments of the present invention.

Before any embodiment of the invention is explained in detail, it is to be understood that the present invention is not limited in its application to the details of construction and the arrangements of components set forth in the following description or illustrated in the drawings. The present invention is capable of other embodiments and of being practiced or being carried out in various ways. Also, it is to be understood that the phraseology and terminology used herein is for the purpose of description and should not be regarded as limiting.

### DETAILED DESCRIPTION OF THE INVENTION

The matters exemplified in this description are provided to assist in a comprehensive understanding of various exemplary embodiments disclosed with reference to the accompanying figures. Accordingly, those of ordinary skill in the art will recognize that various changes and modifications of the exemplary embodiments described herein can be made without departing from the spirit and scope of the claimed invention. Descriptions of well-known functions and constructions are omitted for clarity and conciseness. Moreover, as used herein, the singular may be interpreted in the plural, and alternately, any term in the plural may be interpreted to be in the singular.

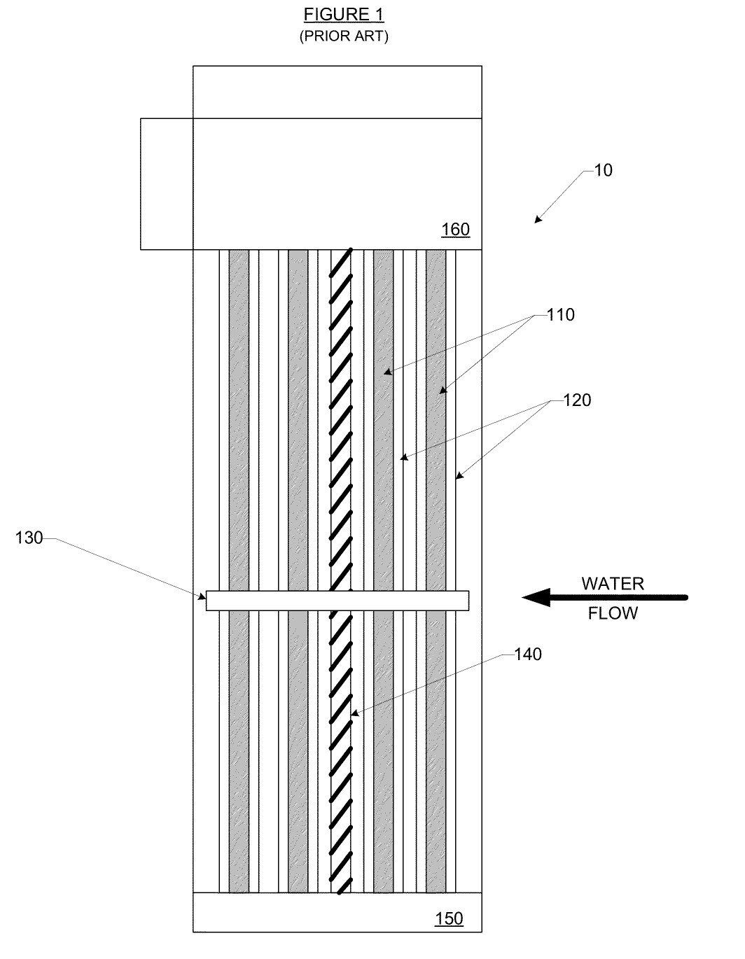

With reference to FIG. 1, an ultraviolet disinfection module 10, as may be known in the prior art will be discussed. UV disinfection module 10 may comprise, among other components, one or more UV lamps 110 that may extend between two headers 150, 160. The lamps 110 may be positioned within a transparent jacket 120, which may be made from UV transparent or resistant materials, such as but not limited to quartz.

In operation, water or wastewater (or any fluid to be treated, including air or other gases) may travel through the UV lamps 110. For example, the water or wastewater may travel in a direction parallel to that of the cleaning plate 130 and therefore pass around and/or between the one or more UV lamps 110. As the water or wastewater passes around and/or between the one or more UV lamps 110, the UV light emitted by the lamps 110 may disinfect or otherwise treat the water or wastewater.

In addition, the UV disinfection module 10 may comprise a cleaning plate 130. A movement device 140—for example, a threaded rod or rotatable screw or screw, a hydraulic piston, an electric or fluid motor, magnetic, a chain drive or other rotary device, etc. may also be present. The movement device 140 may be disposed such that it can effectuate movement of the cleaning plate 130 from one header 150 to the other header 160. Note that the cleaning plate 150 may or may not contact each header 150, 160, but rather may travel substantially between the two.

The Cleaning plate 130 may include a number of holes or orifices aligned with the UV lamps 110 and jackets 120, such that as the cleaning plate 130 travels between the headers 150, 160, the UV lamps 110 and jackets 120 pass through the holes or orifices.

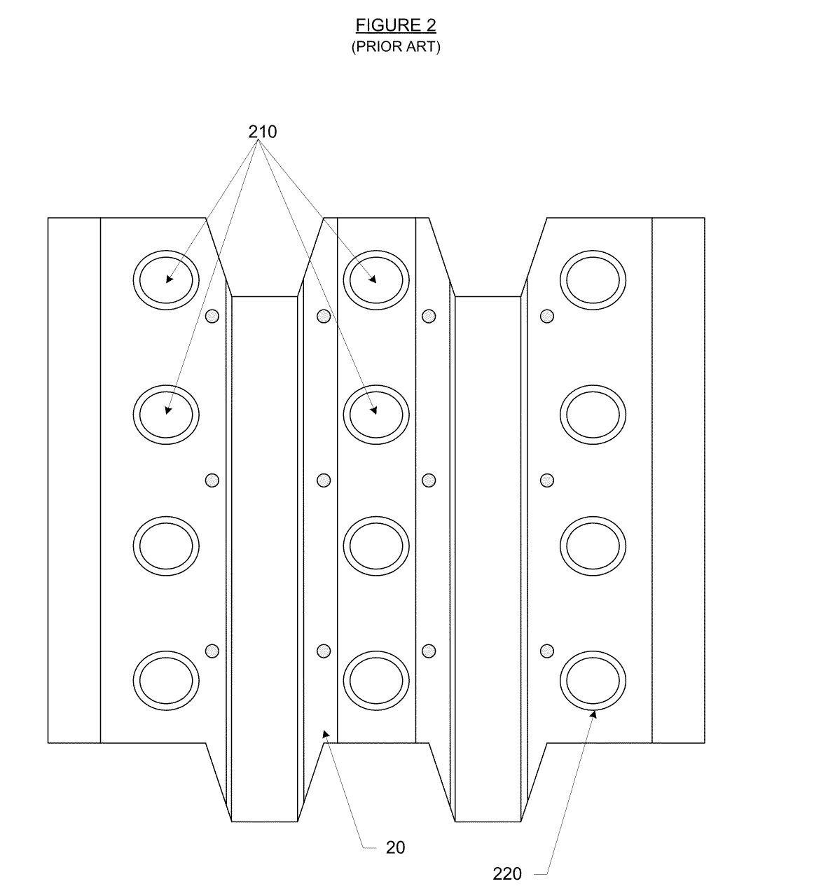

With reference to FIG. 2, a cleaning plate 20 as may be generally known in the prior art is depicted. Cleaning plate 20 may comprise a plate surface with one or more holes or orifices 210 therein. As noted above with regard to FIG. 1, these holes or orifices 210 may be positioned to align with the UV lamps and jackets utilized by the UV disinfection module. In addition, the cleaning plate 20 may comprise a cleaning apparatus 220 that encircles or surrounds each hole or orifice 210. The cleaning apparatus 220 may comprise a wiper, a brush, a squeegee, or any other type of cleaning device. The cleaning apparatus 220 may remove build-up and scale present on the lamps and/or jackets through either mechanical contact (e.g., scrubbing, rubbing, or scraping, etc.), or by other means as may be known in the art (chemical wiping, pressure washing, etc.). In order to provide effective cleaning, the cleaning apparatus 220 generally encircles or surrounds each hole or orifice 210 so that each portion of the UV lamp or jacket is cleaned. In addition, the cleaning apparatus 220 may be sized such that the mechanical interaction between the cleaning apparatus 220 and the UV lamp or jacket is proper to ensure proper cleaning For example, if the cleaning apparatus 220 is a rubber wiper, the rubber wiper 220 will be sized such that as the cleaning plate 20 passes between each header (as discussed above), the rubber wiper 220 will be in sufficient contact with the UV lamp or jacket to effectuate cleaning If the cleaning apparatus 220 is a bristle brush, for example, the sizing of the cleaning apparatus 220 around the hole or orifice 210 may vary.

As noted above, such prior art systems have several drawbacks and disadvantages. For example, it can be very difficult and time consuming to replace the cleaning apparatus 220. Cleaning apparatus 220 may be replaced after wear and erosion of the cleaning apparatus 220 reduces its effectiveness, or may be replaced in order to adapt the cleaning apparatus 220 to the specific conditions of the water or wastewater the UV disinfection unit is treating. For example, the characteristics of some water or wastewater may result in scale accumulating on the UV lamps and jackets, which may require a stiff rubber wiper for removal. The characteristics of other water or wastewater treated may result in a layer of sludge or film accumulating on the UV lamps and jackets, which may require a bristle brush for removal.

Therefore, in accordance with some embodiments of the present invention, a cleaning device that is split into at least two (2) components will now be discussed. In accordance with some embodiments of the present invention, the system may comprise a horizontal UV system, such that the water or wastewater may flow substantially parallel to the UV lamps and jackets and substantially perpendicular to the cleaning plate. It is also contemplated that the water or wastewater may flow in any orientation to the UV lamps and jackets, such as diagonal, at an angle, etc. For example, the system may be an inclined system, in which the UV lamps may extend into the flow of the water or wastewater at an angle. Such orientations may be advantageous since less depth of water or wastewater may be needed while providing sufficient exposure to the UV lamps.

Moreover, in accordance with some embodiments of the present invention, the cleaning plate may not be a plate, but rather may comprise a non-flat surface. For example, the cleaning plate may comprise various steps resulting in rows of wipers at different planes or elevations. As another example, the cleaning plate may not be a “plate” at all, but comprise a system in which the wipers may be secured on cylinders which, in turn, are interconnected. The cleaning plate may also comprise a rigid mesh or other type of framework. A cleaning plate that does not comprise a solid planar surface may be more desirable in horizontal UV systems.

It is additionally contemplated that the present invention may be utilized in pressurized or non-pressurized closed vessel UV reactors. In such closed vessel UV reactors, the axis of the UV lamps may be oriented substantially parallel or substantially perpendicular to the direction of the flow of the water or wastewater being treated. Alternatively, the axis of the UV lamps may be at any angle from the direction of the flow.

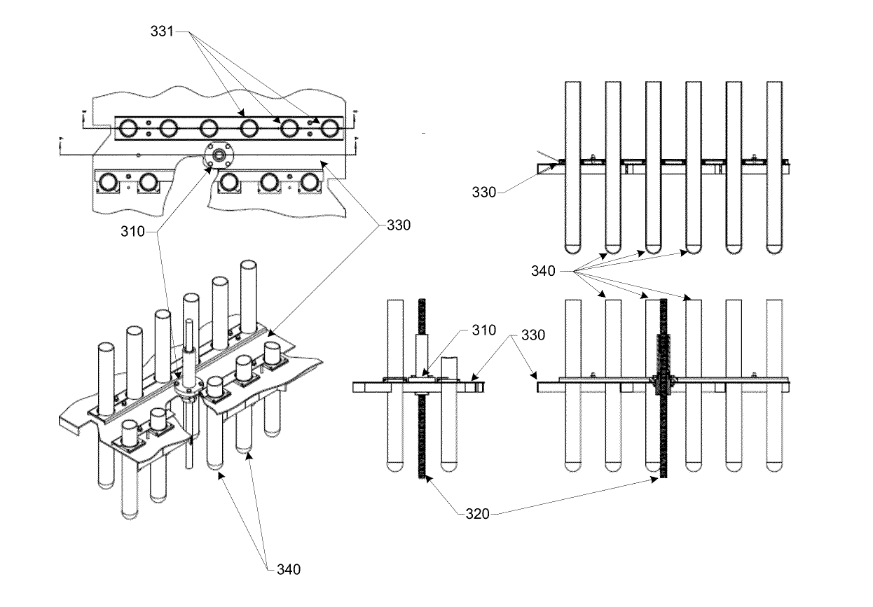

With reference to FIG. 3, a UV disinfection system with a split drive nut 310 in accordance with some embodiments of the present invention will now be discussed. Note that while the system discusses a split drive nut that is in engagement with a threaded rod or rotatable screw, the concept of the present invention is at least in part the split characteristic of the attachment between the cleaning plate and the motion imparting device. Accordingly, while a split drive nut is discussed, the same approach may be used for a split attachment connecting a cleaning plate to a hydraulic cylinder, chain drive, and/or any other type of motion imparting device.

The UV disinfection system may comprise a split drive nut 310, a threaded rod or rotatable screw or other motion imparting device 320, a cleaning plate 330, and one or more UV lamps or jackets 340. The cleaning plate 330 may be a planar element, or as discussed above, may comprise a mesh or framework. The cleaning plate 330 may comprise on or more orifices 331 that are aligned with the one or more UV lamps or jackets 340, such that when the cleaning plate 330 is moved by the threaded rod or rotatable screw or motion imparting device 320, the one or more UV lamps or jackets 340 pass through the orifices 331.

The split drive nut 310 may be attached to the cleaning plate 330 in a removable manner, and may be attached or otherwise engaged to the threaded rod or rotatable screw or motion imparting device 320. In the case of threaded rod or rotatable screw, the split drive nut 310 may include internal threads that may be engaged with external threads of the threaded rod or rotatable screw 320. In the case of a different type of motion imparting device 320, such as a hydraulic piston, the split drive nut 310 may be rigidly attached to an end of the motion imparting device.

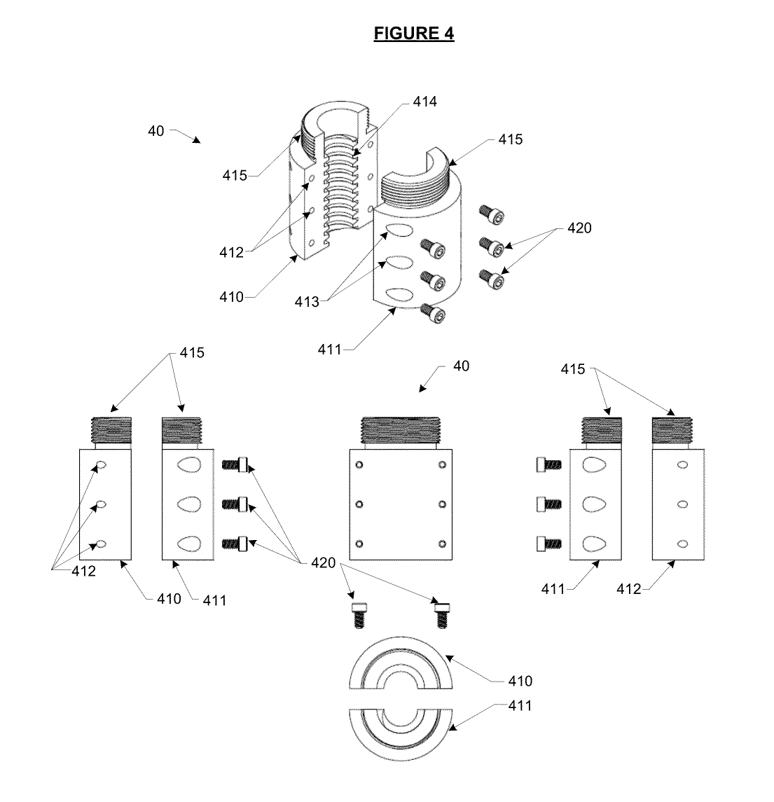

With reference to FIG. 4, a split drive nut 40 in accordance with some embodiments of the present invention will now be discussed. The split drive nut 40 may comprise a first portion 410 and a second portion 411, which, when connected, form the split drive nut 40. Note that while the discussion herein discusses a split drive nut in two (2) portions, it is anticipated that the split drive nut can be any number of portions (e.g., 2, 3, 4, etc.). The first portion 410 and the second portion 411 may be connected through the use of attachment screws 415. Attachment screws 415 may be inserted into openings 413 in the second portion 411 and then screwed into internal threads in openings 412 in the first portion. The openings 413 may be countersunk such that the heads of attachment screws 415 are not visible once the split drive nut is assembled. Note that openings 413 in the second portion may be sized such that the attachment screws may pass freely through the opening without engaging the threads of the attachment screws 420. However, it is contemplated that the attachment screws may be screwed into and in threaded engagement with both the first portion 410 and the second portion 411.

Each of the first portion 410 and the second portion 411 of the split drive nut 40 may also comprise internal threads 414, which may be cut into the inner diameter of an opening running the length of the portions 410, 411. Note that when assembled, the first portion 410 and the second portion 411 may align such that the internal threads 414 may be continuous throughout the split drive nut 40. Accordingly, once assembled, the split drive nut may be threaded onto—and off of—a threaded rod or rotatable screw or shaft.

In addition, each of the first portion 410 and the second portion 411 of the split drive nut 40 may also comprise an external threaded portion 415, wherein threads may be cut into the outer diameter of each portion 410, 411. Again, note that when assembled the first portion 410 and the second portion 411 may align such that external threads 415 may be continuous throughout the split drive nut 40. Accordingly, once assembled, the split drive nut may be threaded into—or out of—a hole or orifice tapped with the proper thread size. Note that in accordance with some embodiments of the present invention, the external threaded portion 415 may have a smaller diameter than the overall split drive nut 40.

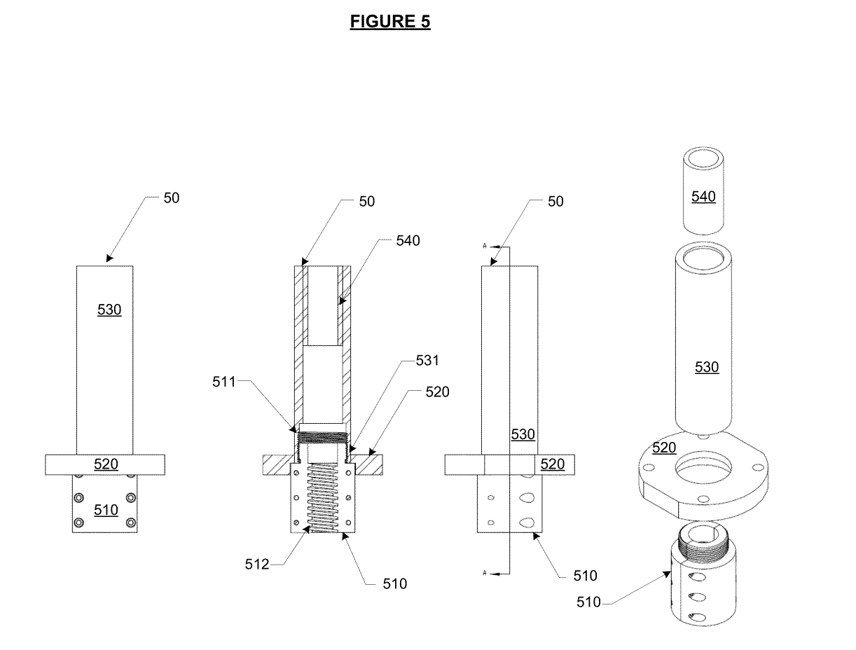

With reference to FIG. 5, a split drive assembly 50 in accordance with some embodiments of the present invention will now be discussed. In general, the split drive assembly 50 may comprise a split drive nut 510 and associated attachment hardware. Associated hardware may include an attachment plate 520, a sleeve 530, and a guide 540. The attachment plate 520 may be used to attach the split drive assembly 50 to a cleaning plate, for example, through the use of holes 521, through which screws, bolts, or other attachment devices may be utilized. Sleeve 530 may be used to attach the split drive nut 510 to the attachment plate 510, as well as to support guide 540.

As discussed above, split drive nut 510 may comprise an external threaded portion 511, as well as internal threads 512. Sleeve 530 may comprise an internally threaded portion 531, sized to accept and engage the threads of the external threaded portion 511 of the split drive nut 510. The split drive assembly 50 may be assembled by inserting the external threaded portion 511 of the split drive nut 510 through an orifice of the attachment plate 520, and then screwing the split drive nut 510 and the guide 530 together, thereby sandwiching the attachment plate 520 between the two. Guide 540 may have an inner diameter sized close to that of the external diameter of the threaded rod or rotatable screw, such that guide 540 may guide the threaded rod or rotatable screw into split drive nut 510 and provide for increased alignment of the same.

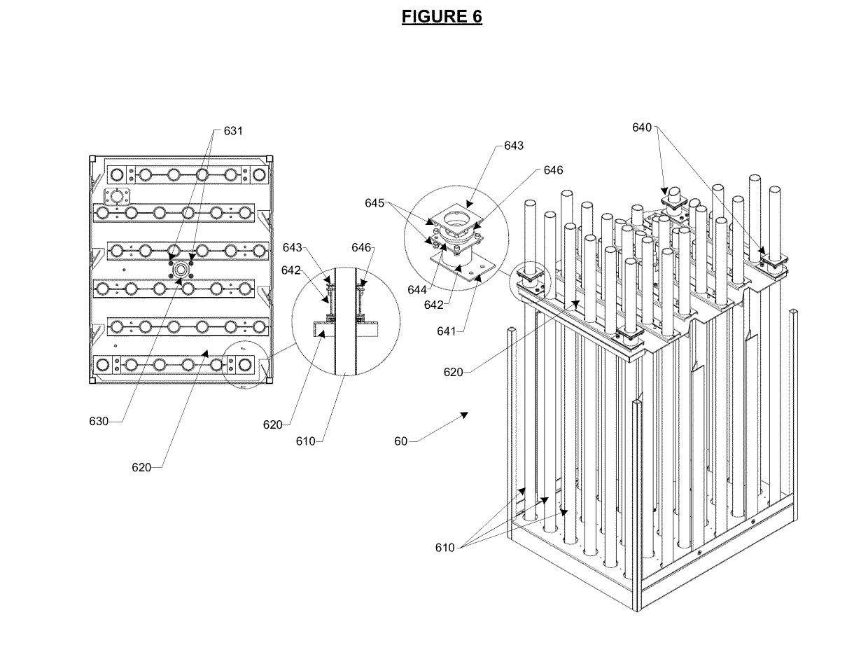

With reference to FIG. 6, a UV disinfection system 60 comprising a split drive nut and sleeve guides, in accordance with some embodiments of the present invention will now be discussed. The UV disinfection system 60 may comprise one or more UV lamps or jackets 610, a cleaning plate 620, a split drive assembly 630, and sleeve guides 640. The cleaning plate 620 may comprise a plurality of orifices sized to receive the UV lamps or jackets 610 as the cleaning plate 620 is moved substantially parallel to the UV lamps or jackets 610. The cleaning plate may be attached to a threaded rod or rotatable screw or other motion imparting device through the use of a split drive assembly 630, as discussed above. The split drive assembly 630 may be attached to the cleaning plate 620 by way of attachments 631, which may comprise screws, bolts, and/or any other type of attachment.

Sleeve guides 640 may be positioned on the cleaning plate 620 in order to assist in maintaining proper alignment of the cleaning plate 620 as the cleaning plate 620 is moved along the UV lamps or jackets 610. Note that while the sleeve guides 640 are shown in the corners of the cleaning plate 620, it is contemplated that any number of sleeve guides may be used in any location on the cleaning plate 620 to provide for proper alignment.

It can be important in UV disinfection systems to maintain proper alignment of the cleaning plate 620 with regard to the UV lamps or jackets 610. While guide rods are often utilized in the prior art, such guide rods may cause unwanted or unnecessary changes in the flow of the liquid through the module. Accordingly, sleeve guides 640 may be used in conjunction with UV lamps or jackets 610 to provide such alignment without altering fluid flow. However, sleeve guides 640 may also be used with regular or common metal shafts or rods as well. The shape, size, and diameter of the sleeve guides 640 may be modified for use on conventional shafts or rods.

Sleeve guides 640 may comprise an attachment plate 641, a sleeve 642, a top plate 643, flange 644, attachment device 645, and an alignment device 646. The sleeve guide 640 may be attached to the cleaning plate 620 through screws, bolts, etc., that may be attached to, or pass through orifices in, the attachment plate 641. Sleeve 642 may be disposed such that UV lamp or jacket 610 may pass through the central opening of the sleeve 642. One end of the sleeve 642 may be attached to the attachment plate 641, and the other end of the sleeve 641 may be attached to flange 644. Alternatively, sleeve 642 and the attachment plate 641 and/or the flange 643 may be an integral component, formed from the same material or permanently attached, for example, through welding the components to form an integral component.

Note that it is contemplated that sleeve 642 may be in a form other than a solid sleeve, for example, to avoid blocking any UV light. For example, the sleeve 642 may comprise one or more rods, grate, or framework so as to avoid blocking any light.

The top plate 643 maybe attached to the flange 644 by way of the attachment device 645 (for example: screws, bolts, etc.). It is also contemplated that top plate 643 may be formed with protrusions which may be inserted into flange 644 (or openings in the flange 644) and then attached, for example through the use of pins, bolts, etc.

An alignment device 646 may be sandwiched between the top plate 643 and the flange 644. The inner diameter of the alignment device 646, for example, a ring, may be sized substantially the same as the outer diameter of the UV lamp or jacket 610. Note that the alignment device 646 may not provide any excessive friction on the UV lamp or jacket 610, but rather may provide sufficient contact with the UV lamp or jacket 610 to maintain the cleaning plate 620 in a proper alignment. For example, the alignment device 646 may comprise an elastic or low friction ring or lip seal, as well as potentially a wiper. Through the use of a plurality of sleeve guides 640, the cleaning plate 620 may be maintained substantially perpendicular to the UV lamps or jackets 610 as the cleaning plate 620 travels the length of the UV lamps or jackets 610.

The length of the sleeve 642 may provide alignment contact with the UV lamp or jacket 610 without altering the cleaning or wiping abilities of any ring or brushes included in or attached to the cleaning plate 620. In addition, the length of the sleeve 642 may provide two contact points with the UV lamp or jacket 610—the alignment device 646 and the wiper or brush associated with the cleaning plate 620.

Through the use of the guide sleeves, the alignment of the cleaning plate may be substantially maintained, thereby reducing the amount of force needed to be applied to the split drive nut to cause the cleaning plate to move along the UV lamps or jackets.

It will be understood that the specific embodiments of the present invention shown and described herein are exemplary only. Numerous variations, changes, substitutions and equivalents will now occur to those skilled in the art without departing from the spirit and scope of the invention. For example, although the guide sleeves are shown with various components having a central orifice through which the UV lamps or jackets pass, the guide sleeves may comprise split components, much like the split drive nut or split wiper assembly as set forth by the applicant. Accordingly, it is intended that all subject matter described herein and shown in the accompanying drawings be regarded as illustrative only, and not in a limiting sense, and that the scope of the invention will be solely determined by the appended claims.

What is claimed is:

1. A split drive assembly for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the split drive assembly operatively connected to a moving device, comprising:
a split drive nut having an internal bore for attachment to the moving device, the drive nut comprising at least two portions attached together around the moving device.

2. The split drive assembly of claim 1, wherein the split drive nut further comprises an external threaded portion.

3. The split drive assembly of claim 2, further comprising:
an attachment plate, used to attach the split drive assembly to the cleaning plate; and
a sleeve, comprising internal threads sized to engage with the external threaded portion of the split drive nut;
wherein the attachment plate is sandwiched between the split drive nut and the sleeve when the split drive nut is inserted into and attached to the sleeve.

4. The split drive assembly of claim 3, further comprising:
a guide, comprising a bore sized substantially similar to a diameter of the moving device, the guide disposed in the sleeve.

5. The split drive assembly of claim 1, wherein the moving device comprises a rotatable screw.

6. The split drive assembly of claim 5, wherein the internal bore of the split drive nut comprises threads sized to engage with the rotatable screw.

7. The split drive assembly of claim 6, wherein the threads of the internal bore of the split drive nut are substantially continuous across the at least two portions.

8. The split drive assembly of claim 1, wherein the moving device comprises a pneumatic or hydraulic piston.

9. The split drive assembly of claim 1, wherein the moving device is selected from the group consisting of: a rotatable screw, a pneumatic piston, a hydraulic piston, a magnetic device, an electric motor, a hydraulic motor, a chain drive, and a cable drive.

10. The split drive assembly of claim 1, wherein the UV disinfection module is a horizontal UV disinfection module, and water or wastewater to be treated flows substantially parallel to at least one UV lamp.

11. The split drive assembly of claim 1, wherein the UV disinfection module is an inclined UV disinfection module, and water or wastewater to be treated flows at an angle to at least one UV lamp.

12. The split drive assembly of claim 1, wherein the UV disinfection module is an inclined UV disinfection module, and the UV lamps are inserted into the flow of water or wastewater at an angle.

13. A split drive assembly for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the split drive assembly operatively connected to a rotatable screw, comprising:
a split drive nut comprising at least two portions attached together, the split drive nut comprising ;
a threaded internal bore sized to engage with the rotatable screw; and
an external threaded portion;
an attachment plate, used to attach the split drive assembly to the cleaning plate; and
a sleeve, comprising
internal threads sized to engage with the external threaded portion of the split drive nut; and
a guide, disposed in the sleeve and comprising a bore sized substantially similar to a diameter of the rotatable screw;
wherein the attachment plate is sandwiched between the split drive nut and the sleeve when the split drive nut is inserted into and attached to the sleeve.

14. A sleeve guide for a cleaning system for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the sleeve guide comprising:
an attachment plate to attach the sleeve guide to a surface of the cleaning plate;
a sleeve extended from the attachment plate; and
an alignment device in contact with a UV lamp, the alignment device attached to the sleeve.

15. The sleeve guide of claim 14, wherein a plurality of sleeve guides is attached to the cleaning plate.

16. The sleeve guide of claim 14, wherein the sleeve guide is disposed on the cleaning plate such that a UV lamp extends through the sleeve guide.

17. The sleeve guide of claim 14, wherein the alignment device comprises a low friction ring or lip seal.

18. The sleeve guide of claim 14, further comprising:
a flange disposed on one end of the sleeve, such that the sleeve extends between the attachment plate and the flange;
a top plate attached to the flange.

19. The sleeve guide of claim 18, wherein the alignment device is sandwiched between the flange and the top plate.

20. A sleeve guide for a cleaning system for an ultraviolet (UV) disinfection module, the UV disinfection module comprising one or more UV lamps extending between one or more headers and a cleaning plate having one or more openings therein arranged to substantially coincide with the one or more UV lamps to permit movement of the cleaning plate along the UV lamps, the sleeve guide comprising:
an attachment plate to attach the sleeve guide to a surface of the cleaning plate;
a flange;
a sleeve extended between the attachment plate and the flange;
a top plate; and
an alignment device in contact with a UV lamp, the alignment device sandwiched between the flange and the top plate.
